(19) United States
(12) Patent Application Publication
(10) Pub. No.: US 2024/0200038 A1
(43) Pub. Date: Jun. 20, 2024

(54) METHOD FOR SEPARATING AND PURIFYING INFLUENZA VIRUS-LIKE PARTICLES (VLPS) USING AQUEOUS TWO-PHASE SYSTEM (ATPS)

(71) Applicant: ZJ Huadi Pharma Group Pharma Development Co., Ltd., Hangzhou (CN)

(72) Inventors: Yang WANG, Hangzhou (CN); Baobin YU, Hangzhou (CN)

(73) Assignee: ZJ Huadi Pharma Group Pharma Development Co., Ltd., Hangzhou (CN)

(21) Appl. No.: 18/483,293

(22) Filed: Oct. 9, 2023

(30) Foreign Application Priority Data
Dec. 15, 2022 (CN) 202211619775.8

(51) Int. Cl. C12N 7/00

(52) U.S. Cl. CPC C12N 7/00; C12N 2760/16123; C12N 2760/16151

(57) ABSTRACT

The present disclosure relates to the technical field of separation and purification of virus-like particles (VLPs), in particular to a method for separating and purifying influenza VLPs using an aqueous two-phase system (ATPS). In the method for separating and purifying influenza VLPs by an ATPS of the present disclosure, a phosphate buffer as a phase-forming salt is mixed with PEG400 to form an aqueous two phase-based extraction system to extract the influenza VLPs. Compared with the prior art, the method for separating and purifying the influenza VLPs has a simple process, which only requires allowing to stand and centrifugation to form an aqueous two phase; and liquids in upper and lower phases can be recycled and reused. The method shows a low cost and environmental friendliness. The extracted influenza VLPs have desirable purity, and a structure and an activity of the influenza VLPs are maintained.

### CROSS REFERENCE TO RELATED APPLICATION

This patent application claims the benefit and priority of Chinese Patent Application No. 202211619775.8, filed with the China National Intellectual Property Administration on Dec. 15, 2022, the disclosure of which is incorporated by reference herein in its entirety as part of the present application.

### TECHNICAL FIELD

The present disclosure relates to the technical field of separation and purification of virus-like particles (VLPs), in particular to a method for separating and purifying influenza VLPs using an aqueous two-phase system (ATPS).

### BACKGROUND

Virus-like particles (VLPs) are highly structured hollow protein particles self-assembled from one or more structural proteins of a certain virus. The VLPs range in size from a few nanometers to hundreds of nanometers and can be produced by expression of viral envelope or capsid proteins in a variety of different cell types. Firstly. VLPs are easily expressed in insect, mammalian, or plant cells, and can be designed to carry a variety of exogenous immune antigens and adjuvants. Secondly, the vast majority of VLPs have particle sizes of 20 nm and 100 nm, such that the VLPs can freely enter lymphatic vessels and be passively transported to the subcapsular region of lymph nodes, and then efficiently taken up by antigen-presenting cells (APCs). Thirdly, VLPs mimics a natural assembly process of viruses during the formation, and thus have similar structure, structural characteristics, and immunogenicity to natural virions. The VLPs can activate APCs such as dendritic cells (DCs) and present these APCs to T cells and B cells, thereby effectively stimulating stronger responses of humoral immunity, cellular immunity, and mucosal immunity. Therefore, VLPs are regarded as a highly excellent vaccine antigen delivery system. Repeated and highly-ordered exogenous epitopes distributed on VLPs can bind multiple times with B cell receptors, thereby stimulating strong B cell immunity and lasting antibody responses. Fourthly, due to the lack of regulatory proteins and infectious nucleic acids, VLPs show no ability to replicate and infect and do not depend on the chicken embryo culture system, thereby exhibiting high safety. VLPs cannot replicate in recipients. However, due to their repeat structures, the VLPs can stimulate the immune system to produce higher cellular and humoral immune responses by recognizing repeat subunits. This brings great convenience to the manufacture and administration of VLPs-based vaccines. Based on the above characteristics, VLPs are an ideal platform for vaccine development and have been widely used in vaccine research, including influenza VLP-based vaccines.

Influenza viruses belong to the Orthomyxoviridae family and can be divided into four types (A, B, C, and D). Influenza viruses A and B can cause seasonal epidemics, influenza virus C generally causes mild disease, and influenza virus D is an emerging influenza virus that can infect cattle and pigs. Influenza viruses are enveloped particles containing a single-stranded, segmented RNA genome. Influenza VLPs are produced by spontaneous assembly of hollow structures composed of structural proteins of influenza virus, and have similar morphology and antigenicity to natural influenza virus. VLPs are genome-deficient constructs and therefore cannot infect cells. The VLPs can be generated using methods similar to those for producing intact viruses. Unlike split or subunit vaccines of influenza viruses, VLPs show a surface antigen presentation that is extremely similar to that of the natural viruses.

Downstream processing of influenza virus typically includes centrifugal clarification, ultrafiltration concentration, column chromatography, and ultracentrifugation purification. In addition, there is a method for purifying viruses from a medium by density gradient centrifugation or continuous flow centrifugation using sucrose, potassium tartrate, and cesium chloride.

The existing influenza VLP technology faces the following problems and defects.
(1) The ultrafiltration concentration has a high antigen recovery rate. However, when this technology increases the content of viruses or VLPs, a host protein is also multiplied, such that a concentration effect is greater than a purification effect on the antigen. Moreover, this technology requires specialized equipment. Therefore, the ultrafiltration concentration can only be used in a primary stage of purification to reduce a volume of the antigen solution.
(2) The column chromatography is a mature technology in the downstream process of protein purification. However, the purification of viruses or VLPs requires specific fillers, and this technology has a high filler cost and the need for matching chromatographic equipment with high prices. (3) The purification of viruses or VLPs by density gradient centrifugation or continuous flow centrifugation not only has cumbersome operations, but also requires corresponding ultrahigh-speed centrifuges or continuous flow centrifuges. Accordingly, this technology shows a high equipment cost and is not conducive to large-scale purification.

Therefore, it is an urgent technical problem to develop a simple, practical, low-cost, and easy-to-operate method for rapid purification of VLPs.

### SUMMARY

In view of this, a technical problem to be solved by the present disclosure is to provide a method for separating and purifying influenza VLPs using an aqueous two-phase system (ATPS). The present disclosure provides a method for separating and purifying the influenza VLPs using the ATPS with simple operation, practicality, low cost, and high extraction purity.

The present disclosure provides an aqueous two phase-based phase-forming agent composition, including a phosphate buffer and a PEG400 solution, where the phosphate buffer includes water, KH₂PO₄, and K₂HPO₄.

Compared with other aqueous two phase-based phase-forming agents, in the aqueous two phase-based phase-forming agent composition provided by the present disclosure, the phosphate buffer has a shorter phase separation time, and the PEG400 solution has an extremely strong hydrophilicity. Since the above two have excellent separation and purification effects when used in combination, the operation steps are further simplified; and this composition is environmental-friendly and easier for large-scale industrial production.

Preferably, in the phosphate buffer, the KH₂PO₄ has a mass fraction of 3.92 wt % to 6.72 wt %, and the K₂HPO₄ has a mass fraction of 10.08 wt % to 17.28 wt %; and
the PEG400 solution includes water and PEG400 with a mass fraction of 14 wt % to 24 wt %.

Within this concentration range, the phosphate buffer is closely matched with the PEG400 solution, and can separate and purify sample viruses more efficiently, thereby obtaining higher effect of virus separation and purification.

In some specific examples, the phosphate buffer includes the water, the KH₂PO₄ with a mass fraction of 5.04 wt %, and the K₂HPO₄ with a mass fraction of 12.96 wt %; and the PEG400 solution includes water and PEG400 with a mass fraction of 20 wt %.

Experiments show that within this concentration range, the phosphate buffer can better cooperate with the PEG400 solution to obtain the best separation and purification effect.

The present disclosure further provides use of the aqueous two phase-based phase-forming agent composition in separation and/or purification of VLPs.

In the present disclosure, the VLPs can be artificially synthesized VLPs of various viruses, such as Norovirus, HPV virus, avian influenza virus, diarrhea virus, hepatitis B virus, porcine circovirus, porcine parvovirus, herpes zoster virus, nCOV-2019, and encephalitis virus. In the examples, influenza VLPs are taken as an example to verify a purification effect. More specifically, H1N1 influenza VLPs are used in the examples of the present disclosure.

Experiments show that compared with other compositions, the aqueous two phase-based phase-forming agent composition is particularly excellent in separating and purifying influenza VLPs. Highly-pure influenza VLPs can be obtained that have both structure and activity preserved.

The present disclosure further provides a method for separating and purifying VLPs in an aqueous two phase, including: separating and purifying a crude extract of the VLPs using the aqueous two phase-based phase-forming agent composition.

Preferably, a process of separating and purifying the crude extract includes:
mixing the crude extract of the VLPs with the aqueous two phase-based phase-forming agent composition, allowing to stand to form a liquid-liquid two-phase system, conducting primary centrifugation to form a liquid-solid-liquid three-phase system, and recovering an intermediate solid phase; and
dissolving the solid phase in a PBS solution, and conducting secondary centrifugation to obtain a solution of the VLPs.

More preferably, the crude extract is separated and purified at 18° C. to 35° C.

In some specific examples, the crude extract is separated and purified at 25° C.; the primary centrifugation is conducted at 3,000 rpm for 5 min, and the secondary centrifugation is conducted at 6.000 rpm for 15 min.

Preferably, a preparation process of the crude extract includes:
constructing an Agrobacterium strain containing a viral recombinant plasmid to infect a plant tissue;
crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

Preferably, the TBE extractant includes water, 10 mmol/L of EDTA, 0.45 mol/L of H₃BO₃, and 0.45 mol/L of a Tris salt.

Compared with other extraction methods, the method for separating and purifying VLPs using an aqueous two phase provided by the present disclosure can complete the separation and purification only through simple operations, can obtain high-purity influenza VLPs. and is more suitable for and extended to industrial production.

In the method for separating and purifying influenza VLPs by an ATPS of the present disclosure, a phosphate buffer as a phase-forming salt is mixed with PEG400 to form an aqueous two phase-based extraction system to extract the influenza VLPs. Compared with the prior art, the method for separating and purifying the influenza VLPs has a simple process, which only requires allowing to stand and centrifugation to form an aqueous two phase; and liquids in upper and lower phases can be recycled and reused. The method shows a low cost and environmental friendliness. The extracted influenza VLPs have desirable purity, and a structure and an activity of the influenza VLPs are maintained. Moreover, a sample for extraction can be scaled up, and is suitable for promotion and application of large-scale industrial production.

### BRIEF DESCRIPTION OF THE DRAWINGS

FIG. 1 shows Western Blotting detection that different solid-to-liquid ratios affect the extraction of influenza VLPs in Example 1;

FIG. 2 shows a flow chart of aqueous two phase extraction:

FIG. 3 shows an appearance of influenza VLPs after separation in Example 3;

FIG. 4 shows an appearance of influenza VLPs after separation in Example 4;

FIG. 5 shows an SDS-PAGE gel electrophoresis figure and Western Blotting detection of a separated sample in Example 4;

FIGS. 6A-C show schematic diagrams for results in preparing influenza VLPs with a PEG400/phosphate aqueous two phase in Example 5, where FIG. 6A is an appearance of an influenza VLP solution after resuspension; FIG. 6B is an SDS-PAGE gel electrophoresis result of the influenza VLP solution; and FIG. 6C is a Western Blotting result of the influenza VLP solution;

FIGS. 7A-B show schematic diagrams for results in preparing influenza VLPs with a PEG400/ammonium sulfate aqueous two phase in Example 5, where FIG. 7A is an SDS-PAGE gel electrophoresis result of an influenza VLP solution; and FIG. 7B is a Western Blotting result of the influenza VLP solution:

FIG. 8 shows an appearance of an influenza VLP solution after resuspension in Example 6;

FIGS. 9A-C show schematic diagrams for results in Example 7, where FIG. 9A is an appearance of an influenza VLP solution after resuspension; FIG. 9B is an SDS-PAGE gel electrophoresis result of the influenza VLP solution; and FIG. 9C is a Western Blotting result of the influenza VLP solution;

FIG. 10 shows a transmission electron microscopy (TEM) image of the influenza VLPs; and

FIG. 11 shows hemagglutination results of the influenza VLPs.

### DETAILED DESCRIPTION OF THE EMBODIMENTS

The present disclosure provides a method for separating and purifying influenza VLPs using an ATPS. Those skilled in the art can learn from contents of this disclosure and appropriately improve process parameters. Of particular note, all similar substitutions and alterations will be apparent to those skilled in the art, and they are all deemed to be included in the present disclosure. Methods and uses of the present disclosure have been described through preferred embodiments, and relevant personnel can obviously modify or appropriately change and combine the methods and uses described herein without departing from the content, spirit and scope of the present disclosure to implement and apply the technology of the present disclosure.

The test materials used in the present disclosure are all common commercially available products, which can be purchased in the market. A plant codon-optimized hemagglutinin sequence [Influenza A virus (A/California/165/2019(H1N1))] is synthesized by Tsingke Biotechnology Co., Ltd. Potassium dihydrogen phosphate and dipotassium hydrogen phosphate (AR) are purchased from Shanghai Macklin Biochemical Co., Ltd. PEG6000 and PEG400 (CP) are purchased from Sinopharm Chemical Reagent Co., Ltd. A primary antibody is Influenza A Virus HA Antibody. Rabbit Mab, from Sino Biological Inc. A secondary antibody is Goat anti-Rabbit IgG-HRP antibody, from Huabio.

A method for rapidly separating and purifying influenza VLPs using an ATPS includes the following steps:

### A. Preparation of a Crude Extract Containing Influenza VLPs:

A1. The plant leaves are infected with Agrobacterium EHA105 containing an influenza HA gene plasmid, and the leaves are harvested after 3 d. The plant leaves to be separated and purified to obtain influenza VLPs are crushed in a TBE extractant composed of EDTA, boric acid (H₃BO₃), and a Tris salt, and fully mixed and extracted to obtain an extract.
A2. The insoluble matters in the extract obtained in A1 are filtered off, and a filtrate obtained after filtration is centrifuged at 4° C. to obtain a crude extract of the influenza VLPs.
   B. Separation and Purification of the Influenza VLPs with an ATPS:
B1. The crude extract of the influenza VLPs is added to an ATPS, mixed thoroughly, and allowed to stand to obtain clear upper and lower phases to form a liquid-liquid two-phase system, and then subjected to centrifugation to form a liquid-solid-liquid three-phase system, and an intermediate solid phase is recovered.
B2. The solid phase in step B1 is dissolved with a PBS solution, centrifuged, and insoluble matters are removed to obtain a solution of the influenza VLPs.

### C. Detection of Influenza VLPs:

C1. A certain amount of the solution of the influenza VLPs is added with protein Loading, boiled for 10 min, centrifuged at 12,000 rpm for 10 min, and an obtained supernatant sample is detected by SDS-PAGE gel electrophoresis.
C2. After detection by SDS-PAGE gel electrophoresis, the influenza VLPs are subjected to membrane transfer, and a resulting sample is subjected to Western Blotting detection.

### D. TEM Identification of Influenza VLPs

The sample of influenza VLPs after aqueous two phase extraction and identified by SDS-PAGE gel electrophoresis and detected by WB is treated by phosphotungstic acid negative staining for TEM sample preparation. The specific preparation and observation steps are as follows:
D1. 5 μL of the sample is dropped on a 200-mesh copper grid with a carbon film, adsorbed at room temperature for 5 min, and then the remaining solution is carefully removed with filter paper.
D2. At room temperature, the sample on the copper grid is stained with 10 μL of 2% phosphotungstic acid for 5 min, the remaining solution is carefully removed with filter paper, and then the copper grid is put on the filter paper to dry naturally.
D3. An appearance of the VLPs is observed by TEM at a voltage of 120 KV.

### E. Hemagglutination Test of Influenza VLPs:

A hemagglutinin (HA) protein on a surface of influenza virus particles has a structure that recognizes and adsorbs on receptors on the surface of chicken red blood cells, and then produces agglutination of red blood cells. On a 96-well hemagglutination plate, a row of 50 μL of PBS is added as a negative control, and 50 μL of the influenza VLPs are added and mixed well by pipetting; 50 μL of a 1% chicken red blood cell suspension is added to each well, shaken gently, allowed to stand at room temperature for 30 min to 45 min, followed by observing the results.

The present disclosure will be described in detail below with reference to specific examples.

### Example 1 Preparation of Solutions

1. Preparation of a TBE extractant: 10 mmol of EDTA, 0.45 mol of H₃BO₃, and 0.45 mol of Tris salt were added into a beaker, added with 900 ml of pure water, adjusted to a pH value of 8.0 with concentrated hydrochloric acid, and then diluted to IL to obtain the extractant for later use.
2. Preparation of PEG400 and PEG6000 solutions: 80 g of PEG400 or PEG6000 was quantified to 100 g with ddH₂O to obtain 80 wt % each of a PEG400 stock solution and a PEG6000 stock solution, which were adjusted to a pH value of 7.0 with phosphoric acid or sodium hydroxide.
3. Preparation of a phosphate buffer: 11.2 g of KH₂PO₄ and 28.8 g of K₂HPO₄ were dissolved with an appropriate amount of ddH₂O, adjusted to a pH value of 7.0 with phosphoric acid or sodium hydroxide, and quantified with ddH₂O to 100 g to obtain 40 wt % of a phosphate buffer stock solution.
4. Preparation of an ammonium sulfate solution: 40 g of (NH₄)₂SO₄ was heated and dissolved with an appropriate amount of ddH₂O, adjusted to a pH value of 7.0 with ammonia water or sulfuric acid, and quantified with ddH₂O to 100 g to obtain a 40 wt % ammonium sulfate solution.
5. Preparation of a PBS solution: 7.9 g of NaCl, 0.2 g of KCl, 1.44 g of Na₂HPO₄, and 1.8 g of K₂HPO₄ were dissolved in 800 mL of ddH₂O, adjusted to a pH value of 7.4 with hydrochloric acid or sodium hydroxide, and diluted to 1 L with ddH₂O for later use.

### Example 2 Preparation of a Crude Extract Containing Influenza VLPs

### 1. Plant Expression of Influenza VLPs

A plasmid with a codon-optimized HA gene sequence was transformed into Agrobacterium EHA105, cultured in an LB medium at 28° C. 220 rpm for 20 K, and a resulting bacterial solution was collected and resuspended with an MES solution to OD600=0.8-1.0. Tobacco seedlings growing for four weeks were immersed upside down in the bacterial solution, and vacuumized with a vacuum device for 2 min, after the Agrobacterium infected tobacco leaves, the tobacco leaves were harvested after continuing to cultivate for 3 d.

### 2. Preparation of a Crude Extract Containing Influenza VLPs

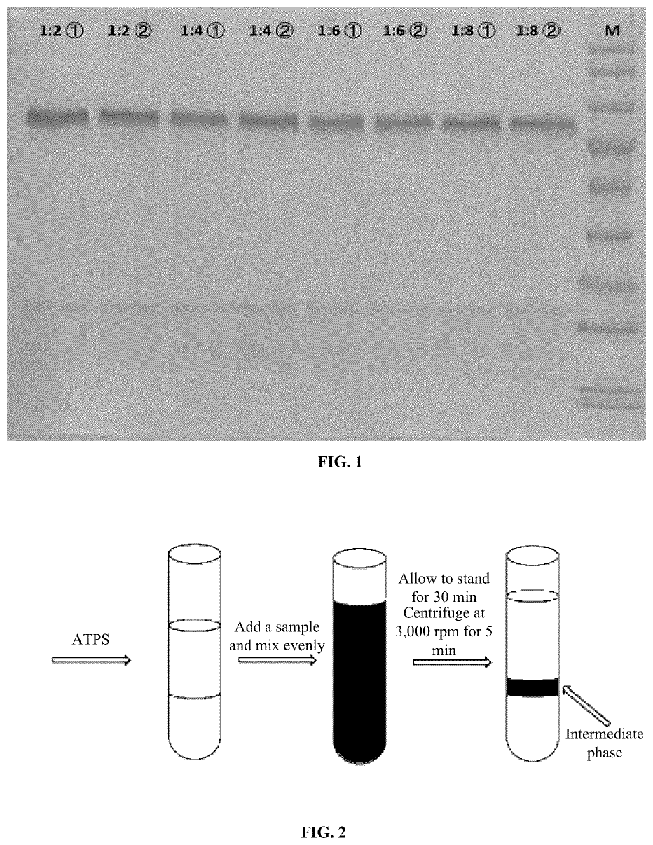

8 identical samples of tobacco leaf powder ground by liquid nitrogen were selected, with 5 g for each sample, were divided into 50 mL centrifuge tubes, a total of 8 tubes, which were marked as 1:2(1), 1:2(2), 1:4(1), 1:4(2), 1:6(1), 1:6(2), 1:8(1), and 1:8 (2) according to solid-to-liquid ratios, including four gradients, with two replicates for each gradient. 10 mL, 20 mL, 30 mL, and 40 mL of the TBE extractant were separately added to the corresponding centrifuge tubes, shaken and mixed well, and digested at 4° C. for 1 h. A filter residue was removed with gauze, then centrifugation was conducted at 6,000 rpm for 15 min at 4° C., a supernatant was collected in each tube, and the 8 tubes of samples were adjusted to a same volume (45 mL) with a TBE solution. 50 μL from each of the samples was taken and tested by Western Blotting, and the results were shown in FIG. 1.

As shown in FIG. 1, within a solid-to-liquid ratio of 1:2 to 1:8, an extraction efficiency of the influenza VLPs by the TBE solution was almost the same. On the basis of taking into account the cost and operability, the solid-to-liquid ratio in actual operation was 1:3.

### Example 3 Comparison Between Preparation of Influenza VLPs with Aqueous Two Phases PEG6000/Ammonium Sulfate and PEG400/Ammonium Sulfate

With a certain mass of the PEG400, PEG6000 stock solution, ammonium sulfate stock solution, and distilled water, 16 wt % ammonium sulfate was prepared as a salt phase, which was mixed with 6 wt %, 8 wt %, and 10 wt % of PEG6000 and PEG400 separately to form six groups of ATPSs with three gradients in each of the two systems, thus extracting the influenza VLPs.

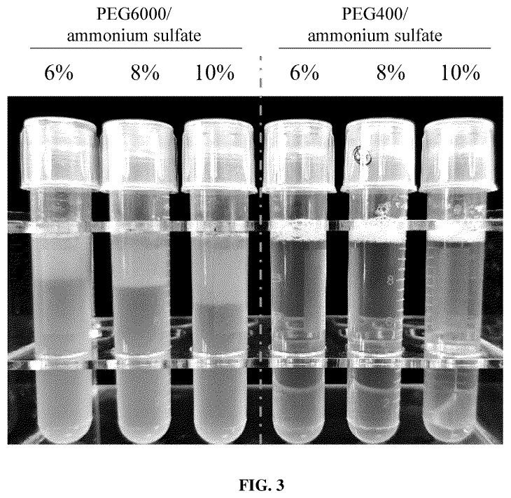

2.5 g of the crude extract containing influenza VLPs was added separately to the above 6 different ratios of ATPSs to form 10 g of ATPSs, where systems not reaching 10 g were supplemented with purified water. The ATPSs each were mixed well by putting upside down or oscillating, allowed to stand at room temperature for 30 min for phase separation, and then centrifuged at 3,000 rpm for 5 min (a flow chart was shown in FIG. 2). The separation of aqueous two phase in these systems was observed, and the results were shown in FIG. 3.

As shown in the figure, the 3 sets of systems of PEG6000/ammonium sulfate could each form a aqueous two phase. However, when influenza VLPs were extracted with ATPS composed of 6 wt % to 10 wt % of PEG400 and ammonium sulfate solutions, the aqueous two phase could not be formed. This indicated that the ATPS composed of low concentrations of PEG400 and ammonium sulfate solutions was not suitable for the separation of influenza VLPs.

### Example 4 Comparison Between Preparation of Influenza VLPs with Aqueous Two Phases PEG6000/Ammonium Sulfate and PEG6000/Phosphate

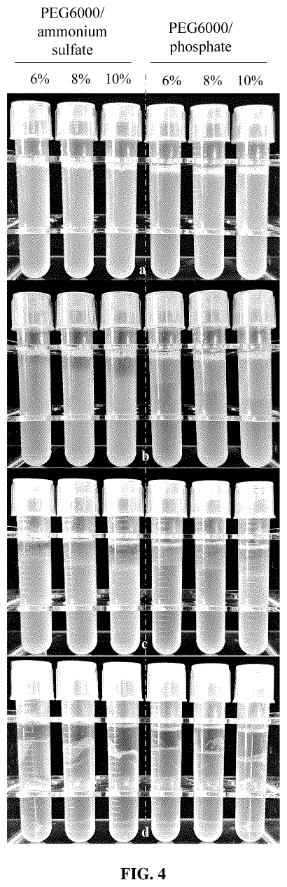

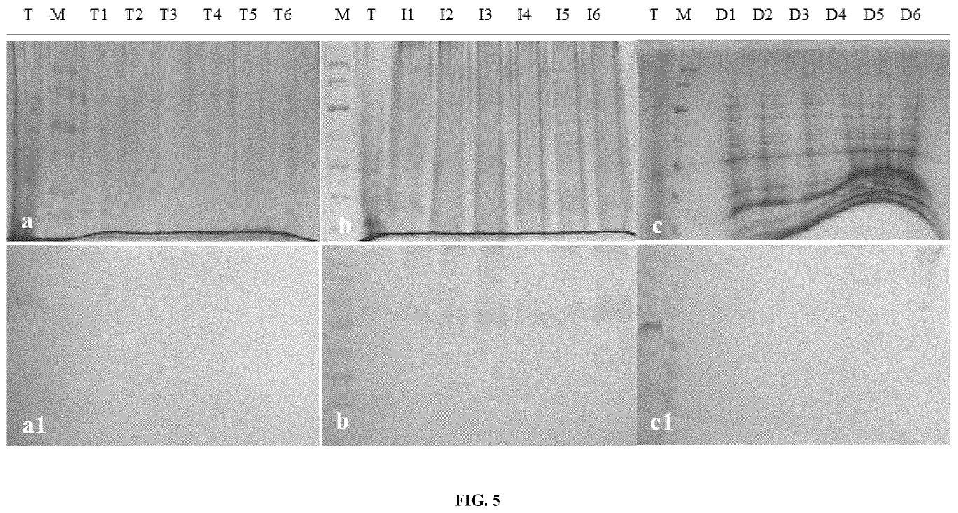

16 wt % of ammonium sulfate or phosphate (phosphate buffer) as a salt phase was mixed with 6 wt %, 8 wt %, and 10 wt % of PEG6000 separately to form six groups of ATPSs with three gradients in each of the two systems, thus extracting the influenza VLPs. The separation of the aqueous two phases was shown in FIG. 4. Each of the aqueous two phases was thoroughly separated by centrifugation, and then an intermediate phase of each system was resuspended and mixed well with 500 μL of PBS, and centrifuged at 6,000 rpm for 15 min to remove insoluble matters to obtain a solution of the influenza VLPs.

The virus was detected by SDS-PAGE gel electrophoresis and Western Blotting. The samples of the intermediate phase of the aqueous two phase of influenza VLPs were collected for SDS-PAGE, and then separated protein bands were transferred to nitrocellulose (NC) membranes, and the NC membranes were rinsed 3 times with a TBST buffer, and then washed with a TBST buffer containing 5% skimmed milk powder for 1 h. The NC membranes were rinsed 3 times with TBST buffer, and an expressed primary antibody was diluted to a working concentration (1:500) with the TBST buffer containing 5% skimmed milk powder, and incubated at room temperature for 1 h. The NC membranes were rinsed 3 times with TBST buffer, then a goat anti-rabbit enzyme-labeled secondary antibody (1:1000) labeled with horseradish peroxidase was added, and incubated at room temperature for 1 h. After rinsing the NC membranes 3 times, a DAB horseradish peroxidase color development solution was added to fully cover the NC membranes, incubated at room temperature until the target bands developed color, and rinsed with pure water twice to stop color development, and dried at room temperature. The results were shown in FIG. 5.

As shown in FIG. 4, the two groups of six systems could form an ATPS. However, it was clearly seen from FIG. 4 b that PEG6000/phosphate was faster than PEG6000/ammonium sulfate in forming phases. FIG. 4 showed the separation results of the aqueous two phase observed at 25° C., and the separation results at 10° C. were similar to those in FIG. 4.

As shown in FIG. 5, M was the protein Marker, T represented the upper phase TOP, I represented the Interphase, and D represented the lower phase Down. T/I/D (1 to 3) represented the upper/intermediate/lower three groups of samples of the 3 aqueous two phase samples of the PEG6000/ammonium sulfate system in FIG. 4, respectively. T/I/D (4 to 6) represented the upper/intermediate/lower three groups of samples of the 3 aqueous two phase samples of the PEG6000/phosphate system in FIG. 4, respectively. FIG. 5A and FIG. 5A1 represented the SDS-PAGE gel electrophoresis and Western Blotting detection of the upper phase samples (T1 to T6). It was seen that the upper phase contained highly less protein (FIG. 5A), and the upper phase did not contain influenza VLPs (FIG. 5A1). FIG. 5B and FIG. 5B1 represented the SDS-PAGE gel electrophoresis and Western Blotting detection of the intermediate phase samples (I1 to I6), it was seen that the intermediate phase contained less protein (FIG. 5B), influenza VLPs mainly existed in the intermediate phase; and as the proportion of PEG6000 increased, the content of influenza VLPs in the intermediate phase was higher (FIG. 5B1). FIG. 5C and FIG. 5C1 represented the SDS-PAGE gel electrophoresis and Western Blotting detection of the lower phase samples (D1 to D6), it was seen that most of the proteins mainly existed in the lower phase (FIG. 5C), the lower phase of the six extraction systems each had a small amount of influenza VLPs.

In summary, the content of influenza VLPs in the intermediate sample 16 of the phosphate system was higher than that of the ammonium sulfate system. FIG. 3 showed that during the sampling, it was found that the sample on PEG6000 upper phase layer had an extremely high viscosity, which was not conducive to small-scale sampling and large-scale sampling, and was also not conducive to the promotion of industrial applications. Moreover, the phosphate system had a short phase separation time (FIG. 4 b), and was more suitable for promotion in industrial applications.

### Example 5 Comparison Between Preparation of Influenza VLPs with Aqueous Two Phases PEG400/Ammonium Sulfate and PEG400/Phosphate

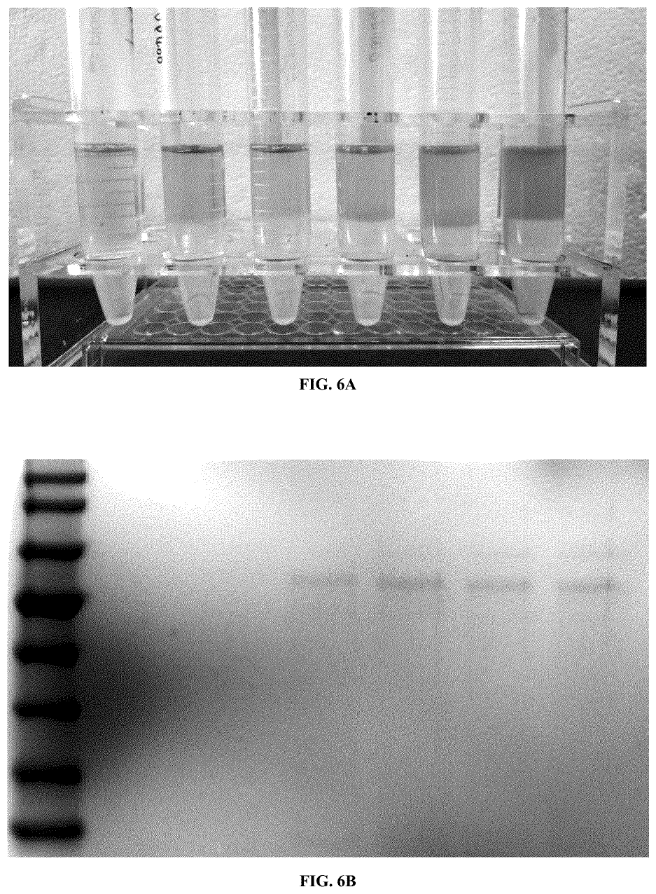

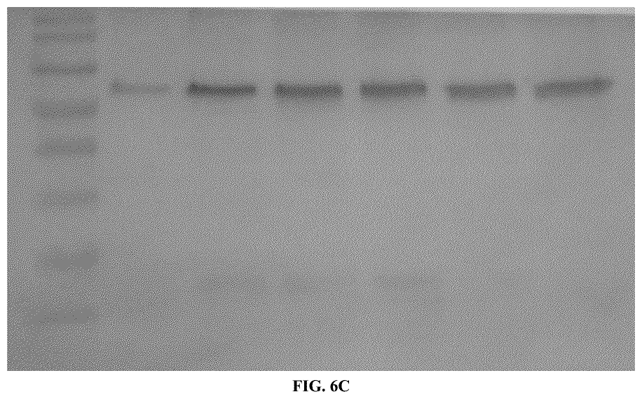

The phosphate buffer with a concentration of 18 wt %, pH=7.0 was separately mixed with the PEG400 with a concentration of 14 wt %, 16 wt %, 18 wt %, 20 wt %, 22 wt %, and 24 wt % to form 6 different systems, which were used to extract the crude extract containing influenza VLPs. Herein, the pH value of a salt phase of the aqueous two phase was 7.0, and it was defined as the pH value of the ATPS was 7.0, while the pH value of a TBE extractant was 8.0, and the pH value of an extract after extracting the total protein of tobacco leaves was about 7.6. After the total protein extract was added to the aqueous two phase, the aqueous two phase was separated, and pH values of an upper phase and a lower phase changed, with a variation range of 7.0 to 8.0. The recovery rate of the intermediate phase and the recovery purity of influenza VLPs were shown in Table 1. The intermediate phase of each system was taken separately, resuspended and mixed well with 500 μL of PBS to obtain a solution of influenza VLPs, and the virus was detected by SDS-PAGE gel electrophoresis and Western Blotting. The results were shown in FIGS. 6A-C.

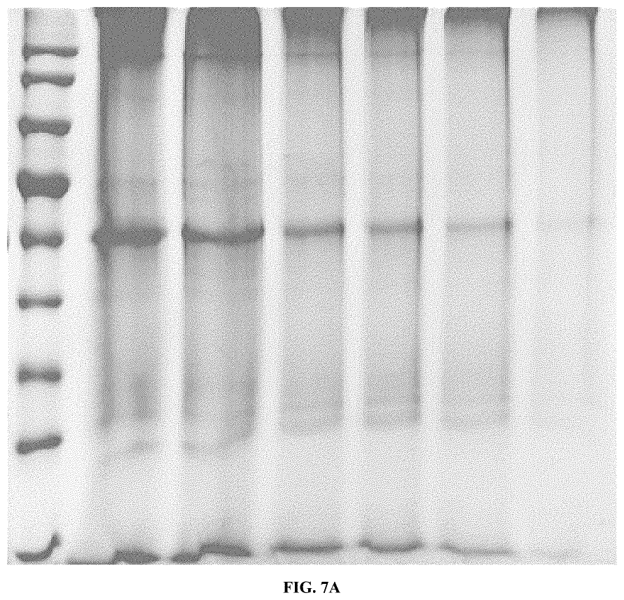

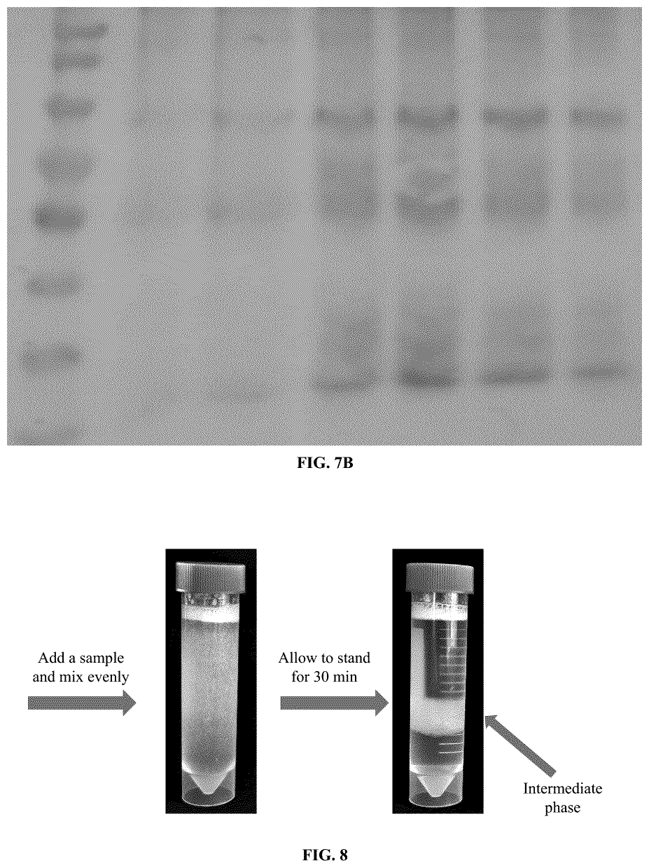

The ammonium sulfate with a concentration of 18 wt % and pH=7.0 was separately mixed with the PEG400 with a concentration of 14 wt %, 16 wt %, 18 wt %, 20 wt %, 22 wt %, and 24 wt % to form 6 different systems, which were used to extract the crude extract containing influenza VLPs. The recovery rate of the intermediate phase and the recovery purity of influenza VLPs were shown in Table 2. The virus was detected by SDS-PAGE gel electrophoresis and Western Blotting, and the results were shown in FIGS. 7A-B.

TABLE 1
Screening conditions for preparation of influenza
VLPs by aqueous two phase phosphate buffer-PEG400

| Phosphate | | | | Purity of |
|---|---|---|---|---|
| buffer | PEG | Concentration | Intermediate phase | influenza |
| (wt %) | (wt %) | multiple | recovery rate (%) | VLPs (%) |
| 18 | 14 | 6 | 5 | — |
| 18 | 16 | 6 | 43.6 | — |
| 18 | 18 | 6 | 78.7 | 83.5 |
| 18 | 20 | 6 | 94.3 | 88.7 |
| 18 | 22 | 6 | 91.2 | 83.3 |
| 18 | 24 | 6 | 85.8 | 79.4 |

TABLE 2
Screening conditions for preparation of influenza
VLPs by aqueous two phase ammonium sulfate-PEG400

| Ammonium | | | | Purity of |
|---|---|---|---|---|
| sulfate | PEG | Concentration | Intermediate phase | influenza |
| (wt %) | (wt %) | multiple | recovery rate (%) | VLPs (%) |
| 18 | 14 | 6 | 2 | — |
| 18 | 16 | 6 | 13.4 | — |
| 18 | 18 | 6 | 49.6 | 23.5 |
| 18 | 20 | 6 | 60.8 | 38.6 |
| 18 | 22 | 6 | 42.5 | 45.1 |
| 18 | 24 | 6 | 28.3 | 64.3 |

An optimal system included the phosphate buffer at a concentration of 18 wt %, pH=7.0 and the PEG400 at a concentration of 20 wt %, and the results showed that clear and specific target bands of influenza VLPs were seen at this time. The influenza VLPs were approximately 60 Kd (FIG. 6C), and the influenza VLPs of intermediate phase had the highest recovery rate of 94.3% and a purity of 88.7% (Table 1). The purification results of ATPS with ammonium sulfate-PEG400 of the same composition were: the highest recovery rate of influenza VLPs in the intermediate phase was 60.8%, and the highest purity was 64.3% (Table 2). In the purification of influenza VLPs, the ATPS of phosphate buffer-PEG400 was much better than that of ammonium sulfate-PEG400. Therefore, the ATPS of phosphate buffer-PEG400 was selected to purify and prepare influenza VLPs. The phosphate buffer with a concentration of 18 wt %, pH=7.0 and the PEG400 with a concentration of 20 wt % were used as an optimal ratio in a one-step method for influenza VLPs by the aqueous two phase.

### Example 6 Scale-Up of 50 g System of Influenza VLPs Prepared by ATPS

According to the optimal system ratio selected under aqueous two phase conditions, the extraction system was enlarged to 50 g. 150 g of the crude extract containing influenza VLPs was added to an aqueous two phase including the phosphate buffer at a concentration of 18 wt %, pH=7.0 and the PEG400 at a concentration of 20 wt %, such that a weight of the entire ATPS was 50 g. The system was mixed well by shaking upside down or oscillating, allowed to stand at room temperature for 30 min for phase separation, and then centrifuged at 3,000 rpm for 5 min to completely separate the two aqueous phase. The intermediate phase was resuspended with 2.5 ml of PBS to obtain a solution of the influenza VLPs (FIG. 8).

### Example 7 Scale-Up of 1,000 g System of Influenza VLPs Prepared by ATPS

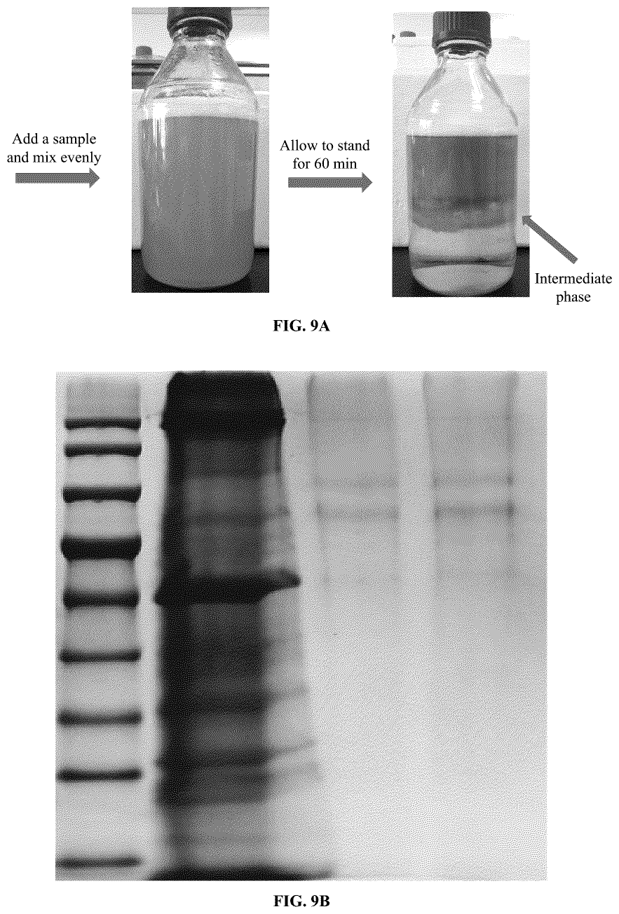

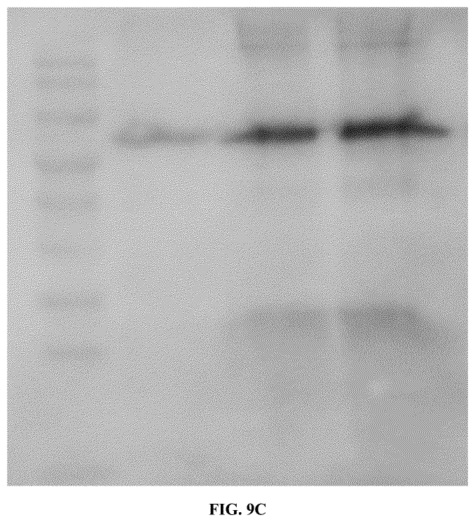

According to the optimal system ratio selected under aqueous two phase conditions, the extraction system was enlarged to 1,000 g. 300 g of the crude extract containing influenza VLPs was added to an aqueous two phase including the phosphate buffer at a concentration of 18 w1%, pH=7.0 and the PEG400 at a concentration of 20 wt %, such that a weight of the entire ATPS was 1000 g. The system was mixed well by shaking upside down or oscillating, allowed to stand at room temperature for 60 min for phase separation, and then centrifuged at 3,000 rpm for 5 min to completely separate the two aqueous phase (FIG. 9A). The intermediate phase was resuspended with 50 ml of PBS to obtain a solution of the influenza VLPs. The phase separation of the aqueous two phase was an inevitable process in this experiment. The duration of this process only affected the efficiency of this method, and did not have a significant impact on the experimental results. The overall trend was that in a same ATPS, the higher the temperature, the shorter the phase separation time was, the larger the ATPS, the longer the phase separation time was. 50 g of ATPS and 1,000 g of ATPS were detected by SDS-PAGE gel electrophoresis (FIG. 9B) and Western Blotting (FIG. 9C). The recovery rates obtained were 93.4% and 94.7%, respectively, and the purities were 89.6% and 88.3%, respectively. It was seen that the ATPS of the present disclosure could effectively scale up, and was stable and easy to operate.

### Example 8 TEM Identification of Influenza VLPs

The sample of influenza VLPs after aqueous two phase extraction and identified by SDS-PAGE gel electrophoresis and detected by WB was treated by phosphotungstic acid negative staining for TEM sample preparation. The specific preparation and observation steps were as follows:
(1) 5 μL of the sample was dropped on a 200-mesh copper grid with a carbon film, adsorbed at room temperature for 5 min, and then the remaining solution was carefully removed with filter paper.
(2) At room temperature, the sample on the copper grid was stained with 10 μL of 2% phosphotungstic acid for 5 min, the remaining solution was carefully removed with filter paper, and then the copper grid was put on the filter paper to dry naturally.
(3) An appearance of the VLPs was observed by TEM at a voltage of 120 Ky.

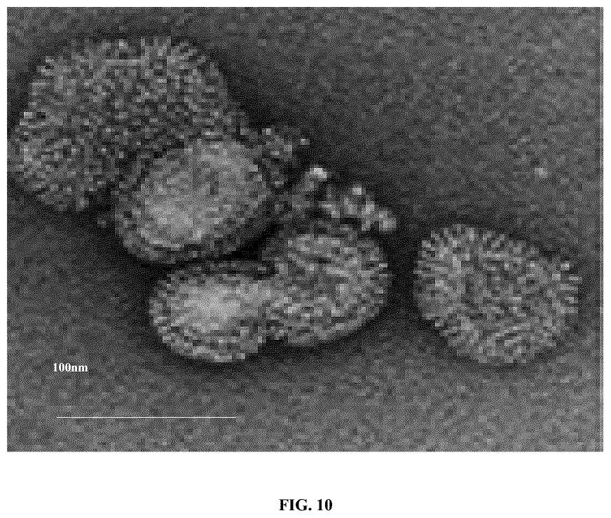

The influenza VLPs prepared by aqueous two phase separation and purification were negatively stained, and then observed by TEM as oval particles covered with spikes and having a diameter of about 100 nm. The results were shown in FIG. 10. This showed that the method for separating and purifying influenza VLPs using an ATPS was efficient and feasible.

### Example 9 Hemagglutination Test of Influenza VLPs

Hemagglutination is a reaction that causes red blood cells to agglutinate in the presence of certain enveloped viruses, such as influenza virus. The hemagglutinin antigen on the surface of the virus interacts with red blood cells and “glues” together to form a lattice structure that makes the red blood cells diffuse. This phenomenon is called hemagglutination. When there is no virus or the virus titer (concentration) is low, the red blood cells in the solution may sink to the bottom of the well and appear as a red dot. A hemagglutinin (HA) protein on a surface of influenza virus particles has a structure that recognizes and adsorbs on receptors on the surface of chicken red blood cells, and then produces agglutination of red blood cells. In this example, the separation and purification of influenza VLPs by ATPS was verified with chicken red blood cells, and the specific test steps were as follows:
1. 25 μL of PBS was separately added to wells 1 to 12 of a 96-well V-type microcoagulation plate.
2. 25 μL of influenza lysed virus (positive control), influenza VLPs, or PBS (negative control) was added to well 1, and mixed evenly.
3. The positive control (first row), influenza VLPs (third row), and negative control (second and fourth rows) were serially diluted (2-fold dilution) on the reaction plate. That is, 25 μL of the suspension was transferred from the first well (stock solution) to the second well (2-fold dilution, recorded as ½), mixed well, and then 25 μL of the suspension was transferred to the third well (4-fold dilution, recorded as ¼), followed by doubling dilution.
4. 25 μL of PBS was added to each well.
5. 25 μL of chicken red blood cell suspension with a volume fraction of 1% was added to each well (the chicken red blood cell suspension was shaken thoroughly before adding).
6. A micro-reaction plate was shaken on a micro-oscillator or the reaction plate was lightly pressed to mix the reactants, allowed to stand at room temperature (about 25° C.) for 40 min, the reaction plate was tilted to 60° C., and whether there were teardrops of red blood cells was observed.

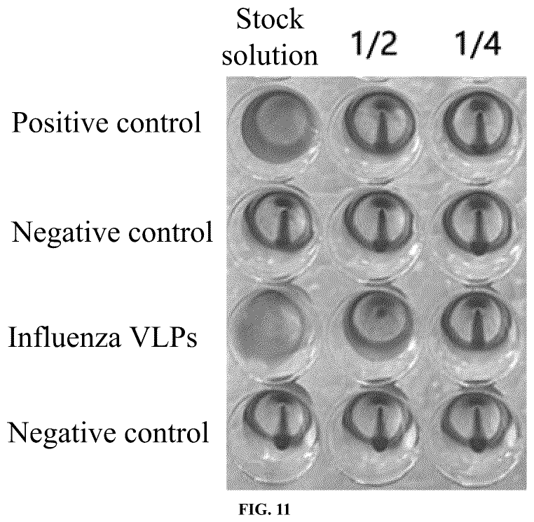

The results were shown in FIG. 11, a hemagglutination titer of the antigen or virus suspension was taken based on a highest dilution factor of complete agglutination and no flow (the second hole in the third row of FIG. 11), and the hemagglutination titer was related to the initial concentration of the antigen. In the negative control, the chicken red blood cells completely sunk to the bottom of the hole in the form of dots. However, in the sample wells of influenza VLPs prepared by aqueous two phase separation and purification, chicken red blood cells were evenly spread on the bottom of the wells in the form of sand, and then settled in the bottom of the wells in the form of dots. This showed that the influenza VLPs separated and purified by ATPS had hemagglutination activity, and also showed that the method for preparing influenza VLPs of the present disclosure was effective and feasible.

It can be seen from the above examples that the present disclosure establishes a method for separating and purifying influenza VLPs using an ATPS for the first time. The method for separating, purifying, and preparing influenza VLPs shows simplicity and practicability, low cost, easy operation, large-scale industrial production, and high extraction rate and purity. The method can obtain higher product purity and recovery rate, and is suitable for industrial promotion and application.

The above described are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. An aqueous two phase-based phase-forming agent composition, comprising a phosphate buffer and a PEG400 solution, wherein the phosphate buffer comprises water, KH₂PO₄, and K₂HPO₄.

2. The aqueous two phase-based phase-forming agent composition according to claim 1, wherein in the phosphate buffer, the KH₂PO₄ has a mass fraction of 3.92 wt % to 6.72 wt %, and the K₂HPO₄ has a mass fraction of 10.08 wt % to 17.28 wt %; and the PEG400 solution comprises water and PEG400 with a mass fraction of 14 wt % to 24 wt %.

3. The aqueous two phase-based phase-forming agent composition according to claim 2, wherein in the phosphate buffer, the KH₂PO₄ has a mass fraction of 5.04 wt %, and the K₂HPO₄ has a mass fraction of 12.96 wt %; and in the PEG400 solution, the PEG400 has a mass fraction of 20 wt %.

4. (canceled)

5. (canceled)

6. A method for separating and purifying virus-like particles (VLPs) in an aqueous two phase, comprising: separating and purifying a crude extract of the VLPs using the aqueous two phase-based phase-forming agent composition according to 1.

7. The method according to claim 6, wherein a process of separating and purifying the crude extract comprises:
mixing the crude extract of the VLPs with the aqueous two phase-based phase-forming agent composition, allowing to stand to form a liquid-liquid two-phase system, conducting primary centrifugation to form a liquid-solid-liquid three-phase system, and recovering an intermediate solid phase; and
dissolving the solid phase in a PBS solution, and conducting secondary centrifugation to obtain a solution of the VLPs.

8. The method according to claim 7, wherein the crude extract is separated and purified at 18° C. to 35° C.; the primary centrifugation is conducted at 3,000 rpm for 5 min, and the secondary centrifugation is conducted at 6,000 rpm for 15 min.

9. The method according to claim 6, wherein a preparation process of the crude extract comprises:
constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and subjecting an obtained extracted product to centrifugation to obtain the crude extract of the VLPs.

10. The method according to claim 9, wherein the TBE extractant comprises water, 10 mmol/L of EDTA, 0.45 mol/L of H₃BO₃, and 0.45 mol/L of a Tris salt.

11. The method according to claim 6, wherein in the phosphate buffer, the KH₂PO₄ has a mass fraction of 3.92 wt % to 6.72 wt %, and the K₂HPO₄ has a mass fraction of 10.08 wt % to 17.28 wt %; and the PEG400 solution comprises water and PEG400 with a mass fraction of 14 wt % to 24 wt %.

12. The method according to claim 11, wherein in the phosphate buffer, the KH₂PO₄ has a mass fraction of 5.04 wt %, and the K₂HPO₄ has a mass fraction of 12.96 wt %; and in the PEG400 solution, the PEG400 has a mass fraction of 20 wt %.

13. The method according to claim 11, wherein a process of separating and purifying the crude extract comprises:
mixing the crude extract of the VLPs with the aqueous two phase-based phase-forming agent composition, allowing to stand to form a liquid-liquid two-phase system, conducting primary centrifugation to form a liquid-solid-liquid three-phase system, and recovering an intermediate solid phase; and dissolving the solid phase in a PBS solution, and conducting secondary centrifugation to obtain a solution of the VLPs.

14. The method according to claim 12, wherein a process of separating and purifying the crude extract comprises:
mixing the crude extract of the VLPs with the aqueous two phase-based phase-forming agent composition, allowing to stand to form a liquid-liquid two-phase system, conducting primary centrifugation to form a liquid-solid-liquid three-phase system, and recovering an intermediate solid phase; and dissolving the solid phase in a PBS solution, and conducting secondary centrifugation to obtain a solution of the VLPs.

15. The method according to claim 13, wherein the crude extract is separated and purified at 18° C. to 35° C.; the primary centrifugation is conducted at 3,000 rpm for 5 min, and the secondary centrifugation is conducted at 6,000 rpm for 15 min.

16. The method according to claim 14, wherein the crude extract is separated and purified at 18° C. to 35° C.; the primary centrifugation is conducted at 3,000 rpm for 5 min, and the secondary centrifugation is conducted at 6,000 rpm for 15 min.

17. The method according to claim 11, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

18. The method according to claim 12, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

19. The method according to claim 7, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

20. The method according to claim 13, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

21. The method according to claim 14, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.

22. The method according to claim 8, wherein a preparation process of the crude extract comprises: constructing an Agrobacterium strain containing a viral recombinant plasmid to infect a plant tissue; crushing and extracting an obtained infected plant tissue with a Tris-Borate-Ethylenediaminetetraacetic acid (EDTA) (TBE) extractant; and
subjecting an obtained extracted product to centrifugation to obtain the crude extract of the VLPs.
